(19) United States
(12) Patent Application Publication
(10) Pub. No.: US 2018/0050128 A1
(43) Pub. Date: Feb. 22, 2018

(54) COMPOSITION, ARTICLES AND METHODS FOR WOUND CARE

(71) Applicant: THE SECANT GROUP, LLC, Telford, PA (US)

(72) Inventors: Peter D. GABRIELE, Frisco, TX (US); Michael S. FLEMMENS, Chicago, IL (US); Jeremy J. HARRIS, Buckingham, PA (US); Jeffrey H. ROBERTSON, Sellersville, PA (US); Kayla WROBLESKY, Schwenksville, PA (US)

(21) Appl. No.: 15/681,180

(22) Filed: Aug. 18, 2017

Related U.S. Application Data
(60) Provisional application No. 62/376,593, filed on Aug. 18, 2016.

(51) Int. Cl. A61L 26/00; C08L 31/06

(52) U.S. Cl. CPC A61L 26/0009; A61L 26/0033; A61L 26/0038; A61L 26/0076; A61L 2430/40; A61L 26/009; C08L 31/06; A61L 2300/404; A61L 26/008

(57) ABSTRACT

Delivery articles for wound and surgical site treatment to prevent adhesion, provide antimicrobial benefits, and provide tissue scaffolding or support. The articles include (1) a dispensing unit or similar apparatus, the dispensing unit being selected from a pressurized or pressurizable apparatus. The dispensing unit contains (2) (a) a PGS resin, optionally including micronized PGS thermoset resin (b) a solvent (c) optionally, a propellant or other dispersant, (d) optionally, a mixture or suspension or dispersion or solution of one or more biologic tissue engineering ECM-compatible biologic components, antimicrobials, drugs, growth enhancers, stimulants, trophic agents, tissues, tissue matrices, and cells, and (e) optionally, one or a combination of structural matrix materials, fibers and fillers, gelatin and collagen.

### RELATED APPLICATIONS

This application claims the benefit of, and priority to, U.S. 62/376,593 filed Aug. 18, 2016, which is hereby incorporated by reference in its entirety.

### FIELD

The present application is generally directed to compositions, articles and methods for spray and ointment formulations comprising biodegradable resins to provide conformable constructs. More particularly, the application is directed to articles and compositions for delivery of oligomeric and polymeric degradable resins, even more particularly poly(glycerol sebacate) (PGS) and oligomeric (glycerol sebacate) (OGS) resins, in wound care and surgical applications for providing conformable constructs including coatings, films, fillers, and the like.

### BACKGROUND

Chronic wounds are gaping losses of viable whole tissue located on the surface of a patient's anatomy. These wounds are complex conditions of the skin and subcutaneous tissue and do not heal in an orderly set of stages and in a predictable manner as with other wounds and can sometimes be characterized by lasting three months or more prior to healing. Wounds can also be considered internal traumatic loss of tissue, as well as iatrogenically caused. Regeneration of the tissue within the subject space requires the regeneration of viable tissue including the subdermal structures, blood vessels, nerves, muscle and fascia, as well as the epidermis.

The wound healing process is actually a regenerative process requiring neovascularization, subcutaneous tissue regeneration and neoepithelialization. Often gaping chronic wounds heal by granularity or scar tissue, impairing normal function and creating pain for the patient. Chronic wound healing is now considered a tissue engineering challenge. One approach is to reconstitute or redevelop the extracellular matrix (ECM) in the wound void space to allow stem cells and vascularization and innervation to populate the wound space. Trophic agents and scaffold materials are believed to be key to developing the ECM. However, unlike traditional tissue engineered constructs, chronic wounds require a conformal system of matrix deposition as a result of the irregularity involved in wound space boundaries and structures.

Polymers of glycerol/sebacic acid (PGS), including both homopolymers and copolymers, have been shown to hold great promise as a bioresorbable material for use in medical and other applications, and would be suitable for wound healing. However, processing challenges have limited the use of PGS for a variety of applications. For example, the neat uncured resinous form of PGS is sticky and difficult to manipulate. Because PGS has a melt temperature of ˜35° C. and the curing process to produce the thermoset elastomer requires temperatures above 100° C., typical conformable constructs must be formed and cured. Thus, the existing forms of PGS preclude its use as an in situ conformable coating or filler, particularly for medical applications.

PGS resins provide benefits that can address unmet needs for providing conformable coatings and fillers for a variety of medical and non-medical applications, and particularly for in situ use in medical clinical applications. The unmet needs in the wound care market include a desire for cost of care reduction through shortening of healing times, as well as clinical shortcomings in areas such as the desire to decrease pain associated with wounds and increase patient comfort, provide better infection resistance, tissue regeneration to aid healing, and to restore function, among other things. Inventions according to this disclosure provide articles, compositions, and methods for addressing these unmet needs by incorporating PGS resin into forms for delivery of conformable constructs, including but not limited to in situ delivery to animal tissue.

### SUMMARY

Exemplary embodiments are directed to articles, compositions and methods using PGS for one or more of ointment, spray and foamed dispersion. The articles, compositions, and methods are useful for a variety of applications, particularly biological tissue applications, and more particularly for use in wound care and surgical applications. The invention provides delivery articles for containing and dispensing PGS compositions and in some embodiments for metering delivery of the compositions. The invention also provides conformable constructs formed by liquidous, spray or foamed dispersion, the conformable constructs useful for coating or filling a target site, which may be delivered in situ or may be formed before application to a target site.

In accordance with the various embodiments, the delivery article includes (1) a dispensing unit or similar apparatus, in some embodiments the dispensing unit being selected from a pressurized or pressurizable apparatus, the dispensing unit containing (2) a composition comprising (a) a PGS resin, optionally including micronized PGS thermoset resin (b) a solvent (c) optionally, a propellant or other dispersant, (d) optionally, a mixture or suspension or dispersion or solution of one or more biologic tissue engineering ECM-compatible biologic components, antimicrobials, drugs, growth enhancers, stimulants, trophic agents, tissues, tissue matrices, and cells, and (e) optionally, one or a combination of structural matrix materials, fibers and fillers, gelatin and collagen. In some embodiments, the treatment composition (2) contains one or more drugs, medicaments, or other biologically and/or pharmaceutically active ingredients that may be incorporated therein for controlled release during subsequent resorption or degradation of the resin due to the PGS surface eroding characteristics.

In some embodiments of the above described compositions, the resin is cross-linked after application, which may be accomplished by any suitable technique including energy application or chemical method. It will be appreciated that one benefit of using non crosslinked resins as described in some embodiments herein is that uncured resin, particular uncured PGS and OGS, will degrade more rapidly than a thermoset, making it more suitable for certain applications such as wound/surgical sites.

Further, and advantageously, compositions that comprise PGS Flour, and PGS flour with resin flour and/or composite formed with any of this, according to embodiments as disclosed herein, also prevent wound contraction that commonly seen in burns using conventional dressings. Without being bound by theory, it is believed that one factor influencing this effect is the slower degradation of these thermosets as compared to traditional wound dressing materials, which enables these embodiments to provide a physical barrier within the wound or surgical site that prevents the contraction.

According to one exemplary embodiment, the composition (2) comprises a resin comprising PGS. In some such embodiments, the resin has an average molecular weight in the range of from about 1,000 to about 50,000 Da and a molar ratio of glycerol to sebacic acid in the range of 0.7:1 to 1.3:1. In some such embodiments, the resin has a molecular weight in the range of from about 15,000 to about 25,000 Da. In some such embodiments, the composition (2) comprises resin comprising PGS, supplemented with one or more of trophic agents and cells delivered by a specialized dressing in a trans-matrix fashion much like a transdermal reservoir.

According to another exemplary embodiment, the composition (2) comprises a mixture including a resin comprising PGS, and a micronized thermoset filler comprising PGS. In some such embodiments, the thermoset filler and the resin each have a molar ratio of glycerol to sebacic acid in the range of 0.7:1 to 1.3:1. In some such embodiments, the thermoset filler has a particle size between 0.5 and 1000 microns. In some such embodiments, the thermoset filler has a particle size less than 250 microns. In some such embodiments, the thermoset filler is present as in a range of from about 10% by weight to about 90% by weight of the mixture. In some such embodiments, the thermoset filler is present in a range of from about 40% by weight to about 70% by weight of the mixture.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows graphical data obtained pertaining to testing embodiments hereof;

FIG. 2 shows graphical data obtained pertaining to testing embodiments hereof;

FIG. 3 shows graphical data obtained pertaining to testing embodiments hereof; and

FIG. 4 shows graphical data obtained pertaining to testing embodiments hereof.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF EXEMPLARY EMBODIMENTS

Exemplary embodiments of the invention provide a system to treat chronic, traumatic and surgical wounds. In accordance with the various embodiments, the system includes providing a conformal wound-bed or surgical site construct in the form of any one more of topical films, tissue in situ films and filler constructs, fibrous constructs and devices, elongate string constructs and devices, and hydrogel constructs. In use, the various delivery articles deliver and facilitate forming, in situ and as controlled by a user of the delivery article, a construct that comprises conformal and consistent, and/or a mixed plurality of components for forming the structure of the construct and for delivering biological actives. As described further herein, it will be appreciated that while the exemplary embodiments are directed to medical applications, particularly in the context of wounds and surgical sites, the inventions hereof are not limited in their use to such applications.

The various embodiments are directed to articles, compositions and constructs comprising PGS for use in wound care applications, including the treatment of both chronic and acute wounds, which may include use in surgical deposition. Advantages of the use of PGS in wound care include its bio-resorbability and degradation to glycerol and sebacic acid, which are natural metabolic byproducts. As a result, PGS chemistry may have a particular benefit in diabetic wound care through cellular fueling resulting from the breakdown of products in direct wound application. Further, the presence of sebacic acid also acts to decrease wound pH, which aides in wound healing, as chronic wounds maintain elevated pH levels prolonging the healing process.

Formulations in accordance with exemplary embodiments may be doped with appropriate trophic agents, stem or somatic cells, as well as other wound care agents. The device and composition of matter may be modified to deliver additional wound space needs such as hemostatic agents, antibiotics, analgesics, active pharmaceutical ingredients (APIs) or other wound care materials such as ointments, alginates, hydrogels, and fillers. The composition may include alginate for exudate absorption, a deodorant, antibiotic, trophic agent, Manuka honey and combinations thereof.

Thus, the embodiments include active components, together with delivery articles for forming the various constructs. In accordance with the various embodiments, the delivery article includes one of a pressurized dispensing unit or similar apparatus containing one of a propellant or other dispersant, and also containing one or a combination of structural matrix materials, fibers and fillers, and also containing one or a combination of biologic or wound healing active comprising a mixture or suspension or dispersion or solution of biologic tissue engineering ECM-compatible biologic components, antimicrobials, drugs, growth enhancers, stimulants and trophic agents, tissues and tissue matrices, and cells.

Exemplary embodiments are not so limited and a variety of different compositions can be formulated, including those containing collagen or other fibrous material to form sprayable hydrogels for use in wound care compositions for emergency field deployment, EMT and emergency hemostasis. Further, spray compositions can be provided as aerosol pre-surgical prophylactic sprays for surgical site infection (S SI) control or pre-deployment treatment protection, for example for direct surgical site spray for SSI prophylaxis and preparation treatment for textile implants immediately prior to implant procedure. Still other embodiments of compositions used in spray applications include PGS doped microparticles for direct application in the form of encapsulated trophic agents and/or analgesics, as well as delivery of hyperthermia targets. It will be appreciated that PGS, in particular, is conformal, particularly when delivered according to embodiments hereof. Further, PGS is angiogenic, non-thrombogenic inherently antimicrobial, non-immunogenic, and nutritional to senescent cells by virtue of delivery of the glycerol by product of its breakdown in vivo. And PGS is acidic, which benefits a diabetic wound that is usually stuck in alkalinity.

Among the applications for which exemplary embodiments of the invention may be employed include a wide variety of medical and industrial applications such as the creation of an implant, or as a lubricant or coating on implants or other devices used in orthopedic, neural, and cardiovascular applications, for example. Other medical applications include use in wound care. Still other medical applications include delivery of substances by subcutaneous injection, biomedical device coating/adhesive/sheets or films for implant device prophylactic peri-operative post-surgical infection control; temporary barriers; any surface where microbial colonization threatens human health or condition; hydrophilic agents; textile treatments; veterinary; wound care; biofilm control; regenerative engineering without antibiotic need, two-part drug delivery, and as a porogen, all by way of example.

While primarily discussed in the context of medical applications and wound care in particular, the principles of the invention may also be applied to controlled release PGS based aerosol or other spray coatings for pesticides, agricultural, and industrial applications, including but not limited to agriculture; construction; water management; surface preservation; architectural preservation; anti-fouling; environmental barriers; wound healing fabric surfaces, treatments, coatings, and controlled release vehicles; food additives, among others, as well as pre-gamma sterilization radiation oxidation sealer. Further, industrial applications include use in degradable paints and inks; food processing to deliver flavor or vitamins; water treatment such as for controlled release of algaecide, pesticide or other treatments; as delayed release fish food, surface protection sanitization; water management; filtration; fabric coating for protection; implantable textiles; prophylactic prosthetic implant coatings; conformal coatings; cosmetics; over the counter pharmaceuticals; and aquaculture, all by way of example only.

Advantages realized with the invention according to the various embodiments is the ability of users to deliver and create constructs using a packaged, pre-dosed and pre-metered system for off-the-shelf fabrication of wound site ECM-like constructs and therapeutic and regenerative dressings. A particular advantage of the compositions and constructs deliverable hereunder is that some embodiments can be delivered with porosity in the deposited construct by using fillers, particularly PGS flour filler. The porous deposited constructs, when used in open tissue such as wounds and surgical sites, can capture exudate and avoid wound and dressing fouling without assaulting the wound and creating pain.

Thus, in accordance with the various embodiments, the invention includes delivery articles for wound and surgical site treatment to prevent adhesion, provide antimicrobial benefits, and provide tissue scaffolding or support. The articles include (1) a dispensing unit or similar apparatus, the dispensing unit being selected from a pressurized or pressurizable apparatus. The dispensing unit contains (2) (a) a PGS resin, optionally including micronized PGS thermoset resin (b) a solvent (c) optionally, a propellant or other dispersant, (d) optionally, a mixture or suspension or dispersion or solution of one or more biologic tissue engineering ECM-compatible biologic components, antimicrobials, drugs, growth enhancers, stimulants, trophic agents, tissues, tissue matrices, and cells, and (e) optionally, one or a combination of structural matrix materials, fibers and fillers, gelatin and collagen.

The relative amounts and particular particle sizes of the PGS and other constituents of the spray composition may be varied depending on the desired end use, thus, as further described herein, in some embodiments, the composition may comprise from about 10% to about 60% by weight PGS resin, about 4% or less by weight surfactant, 3 to 10% by weight other additives, and 40 to 90% by weight solvent. In other embodiments, the percentages of the components may be varied, as further described herein.

Delivery of Sprayable Treatment Compositions to Provide Conformable Constructs

Generally, the bio-degradable oligomers and polymers used according to the invention are incorporated into ointment, spray and foam delivery articles for providing topical films, tissue in situ films and filler constructs, fibrous constructs and devices, elongate string constructs and devices, and hydrogel constructs. The delivery articles, as further described herein below, include foaming and aerosol spray propellants, film formers, one or more resorbable/degradable oligomers and polymers, such as the resin PGS and OGS, and optionally one or more active or inert fiber materials to facilitate formation of the constructs. Also included in some embodiments are various combinations of one or more of biological actives, preservative, antimicrobial and aesthetic components. According to the various disclosed embodiments, the constructs and devices contemplated by the present disclosure for use in wound care include films, bandages, dressings, and other larger, easily manipulated devices as well as smaller, particulate, fibrous, foams, or other amorphous shaped constructs that can be delivered using foam, aerosol or other spray methods.

Generally, the delivery article includes (1) a dispenser, in some embodiments the dispenser being selected from a pressurized or pressurizable apparatus, the dispenser containing (2) a composition comprising (a) a PGS resin, optionally including micronized PGS thermoset resin (b) a solvent (c) optionally, a propellant or other dispersant, (d) optionally, a mixture or suspension or dispersion or solution of one or more biologic tissue engineering ECM-compatible biologic components, antimicrobials, drugs, growth enhancers, stimulants, trophic agents, tissues, tissue matrices, and cells, and (e) optionally, one or a combination of structural matrix materials, fibers and fillers, gelatin and collagen. In some embodiments, the treatment composition (2) contains one or more drugs, medicaments, or other biologically and/or pharmaceutically active ingredients which may be incorporated therein for controlled release during subsequent resorption or degradation of the resin due to the PGS surface eroding characteristics. In some embodiments, the resin is cross-linked after application, which may be accomplished by any suitable technique including energy application or chemical method.

The composition (2) may be designed to be curable to fix the string resin. The composition may also be formulated to provide ancillary benefit with such function as to sealing, deodorizing, absorbent, antibiotic or hemostatic as an example.

In some representative examples the composition (2) is useful as a general liquid surgical site flush to prevent adhesions using liquid, foamed or aerosol delivery, or as a gel implant to facilitate healing and discourage adhesion over time. Accordingly, in some embodiments, a treatment composition (2) may include a resin comprising one or more of PGS and OGS, one or more nonionic surfactant, one or more gelling agents selected from collagen, gelatins, dextrans, and glycans, one or more actives, such as antioxidants, and a buffered aqueous vehicle. Optionally, the composition (2) may further include any one or more of an antibiotic, microspheres with API controlled release, imaging agent, nutrient composition, plasma, organ specific chemokines and cytokines, and other biologics. The formulation may be delivered as a foamed or aerosol delivered construct, or as an ointment or gel. Gel formulation rheology can be controlled by use of solid, surfactant, and gelling agents.

Dispenser

The dispenser may be selected from any of a variety of spray, pump dispensers and squeeze dispensers. Generally, these dispensers all share the features of being hand held, enabling dispensing without any need for the user to directly contact the contained materials, thus minimizing issues with tackiness of the composition. And they are all achieve delivery based on use of pressurized propellants, or delivery of pressure due to mechanical mechanisms, or application of pressure by a device or hands of the user. In some alternate embodiments, a dispenser may be adapted for dispensing gel or ointment based compositions, where the dispenser is a tube or squeeze bottle and the pressure delivered by the users hand or some other squeezing mechanism serves to dispense the contents. In other embodiments, a spray dispenser includes a pressurized can with one or more liquefied case propellants. In other embodiments, a spray dispenser may be used to produce an aerosol by generation of pressure with an actuator. In yet other embodiments, a dispenser may be adapted for introducing foam through mechanical actuation and generation of bubbles in a suitable carrier, as described herein below, or mixing in pressurized gas.

The various compositions contained in dispensers may comprise any of a variety of materials customarily used to facilitate dispensing material from the dispensers. Thus, spray dispensers may include propellants, as further described herein. Likewise, foam dispensers may comprise propellant and may alternatively or in addition comprise mechanisms that facilitate the introduction of bubbles. And other hand held dispensers are adapted for dispensing by squeezing, and may include components that facilitate gliding of the material from the interior of the dispenser.

In one exemplary embodiment, a pressurized aerosol dispenser includes a spinneret-like nozzle having a plurality of via passages to create a shower of string composition.

In another embodiment, a pressurized device with a specialized spinneret-nozzle container device delivers the composition as a continuous or sputtered stream to create an ECM like matrix mat within the wound space. The nozzle design can be modified to change spray pattern and string porosity based on wound type such as lacerations, abrasions, traumatic tissue loss, and burns or even a tissue caulking adhesive.

The ability to apply treatment compositions according to the invention using a spray dispenser avoids physical contact between the user and any tools and the affected area, which can reduce irritation while still permitting application of a substantially uniform, thin layer.

Accordingly, advantages of spray applications include the ability to provide and use them in an easy, convenient manner and without risk of contamination or exposure of unused material, which remains in the dispenser. Thus, the embodiments hereof enable economic delivery, portability, conformal therapy, accessibility to wound irregularities.

Yet another advantage is that the nozzle design can be adjusted to permit and vary aeration thus porosity of the construct formed to provide more space and surface area for remodeling of the tissue during healing, and for colonizing cells that may be included as a biologic in the treatment composition.

Constructs

As previously mentioned, in some embodiments compositions may be used to create constructs in the form of fillers and mats useful as dressings, bandages and potentially skin substitutes/grafts to apply to a wound or surgical incision site. Generally, these constructs may have a thickness ranging from about 1 to about 200 μm, and in some embodiments from about 50 to about 100 μm for film-like constructs and up to about 1-5 mm, preferably 2-3 mm, which may be considered more mat-like. These constructs can be applied to absorb exudate, moderate wound pH, eliminate bacteria already present and/or scavenge matrix metalloproteinases, while also forming an antimicrobial barrier between the wound and the environment.

String Constructs

In one exemplary embodiment, a fibrous string construct includes one or more of micro-string-like fibers, a surfactant additive or appropriate additive for providing phase compatibility, an agent to promote fiber-foaming or poration for closed or open cell expansion of string resin. Thus, the delivery article may include a solvent or propellant in which the composition and additives are suitable for expelling or extruding the composition from the container, and for fiber formation.

Among the advantages of exemplary embodiments is the conformal delivery of an ECM/wound space dressing-in-a-bottle using a string-stream method of wound space filling and deposition and creating a construct out of materials that promote the healing process. Such a wound care product can shorten the healing time as a result of encouraging regeneration and should address the chronic wound care need to reduce the cost of treatment. Another advantage is to provide a simple technology that allows for outpatient treatment.

Another advantage is that the use of a spray string method of applying/forming an in situ conformal construct as a one-step delivery process. This in turn may stimulate and direct the tissue edges to proliferate. For instance, in some large wounds it may be preferred to expand the boundaries of the wound space gradually rather than merely filling the gap and redressing the hole wound space periodically.

In still other embodiments, the spray string composition may be used in forming one or more of a hemostatic deposition device, bone void filler or for deep wound tissue repair, and combinations thereof.

Spray Delivery Articles for Preparing Constructs

In some embodiments, resin with one or more additional components selected from fillers, fibers, and biological actives may be dispersed in a carrier for aerosol or other spray-based application. In some particular embodiments, one or more of PGS resin and micronized thermoset PGS, one or more biological actives, and optionally one or more surfactants, fibers or powder particles may be dispersed in a carrier for aerosol or other spray-based application. According to these various embodiments, the articles provide doped PGS antimicrobial topical and internal composition sprays for wound care as well as surgical use using PGS as a carrier for the biologic and as a structural component of a construct when dispensed.

In an exemplary embodiment, the composition combines a PGS resin based delivery article along with active ingredients, surfactant, additives (such as film formers or deodorants), co-solvent and optionally a propellant that include a conformable string or fiber-like extrudate delivered as from a pressurized applicator to create a fiber drop-down, non-woven mat of a ECM-like composition doped with trophic agents and or stem cells. Under the control of the user, the construct formed can be applied to a wound or surgical site to provide a conformal filler and/or coating that is within or covers the wound bed. A fiber dispensing pattern may be uniform of non-woven accumulation of mass. In one exemplary embodiment, a composition includes a 50% solution of PGS in ethanol with dimethyl ether (DME) propellant. Below is an exemplary formulation:

| | % w/w | Grams |
|---|---|---|
| 20% active PGS: | | |
| PGS (50% in EtOH) | 40 | 24 |
| DME | 60 | 36 |
| | 100 | 60 |
| 30% active PGS: | | |
| PGS (50% in EtOH) | 60 | 37.2 |
| DME | 40 | 24.8 |
| | 100 | 62.0 |
| 40% active PGS: | | |
| PGS (50% in EtOH) | 80 | 52.0 |
| DME | 20 | 13.0 |
| | 100 | 65.0 |

In another exemplary embodiment, the composition includes a solution of PGS in ethanol with a suitable propellant such as dimethyl ether and one or more growth factor biologics. Thus, the article can produce, in one example, a construct comprising a poly(glycerol sebacate) (PGS)/fibroin/collagen ‘vehicle-extrudate’ modified with encapsulated vascular endothelia growth factor (VEGF) and/or nerve growth factor (NGF) to form a conformal construct scaffold in the wound bed.

In yet another exemplary embodiment, a spray composition may comprise a 2-part urethane consisting of PGS plus hexamethylenediisocyante (HMDI) from a 2 chamber aerosol unit, wherein a first chamber would contain PGS and the other chamber would contain HMDI. At the nozzle the two materials would combine creating a reaction between the PGS and HDMI to form a polyurethane PGS.

As further described herein above, such treatment composition may be provided in an aerosol article with suitable propellant and carrier components for delivering a string-like extrusion from the sprayer.

Foam Delivery Articles for Preparing Constructs

In still other embodiments, one or more of PGS resin and micronized thermoset PGS, one or more biological actives, and optionally one or more surfactants, fibers or powder particles may be dispersed in a carrier for foam-based application.

In accordance with the disclosure, the foam based delivery article may be an aerosol dispenser with a propellant. In other embodiments, the delivery article may be a pump foamer that is typically manually actuated, and comprises a spring-mounted piston tube that typically reciprocates vertically in an up and down motion within a air and liquid holding cylinder. As generally known in the foam pumping art, the piston tube and an upper and relatively larger portion of the cylinder engage to act as an air pump, while a smaller lower portion of the cylinder and the piston tube act as a liquid pump. The liquid and air pumps are synchronized by the common piston mechanism, and the pumping action is controlled with check valves that regulate entry of liquid from the reservoir into the liquid cylinder, and entry of air into the air cylinder, and discharge of liquid from the liquid cylinder to a liquid/air mixing chamber. The liquid/air mixing chamber also includes a homogenizer. The foam is discharged as a uniform non-pressurized aerated foam through the dispensing head of the dispenser. Some pump heads include a mesh screen, such as a nylon mesh for influencing foam consistency. Of course it will be appreciated that the above description represents an example of a pump dispenser that may be selected in the art.

According to various embodiments of the present invention, a dispensing article in the treatment composition may include one or more emulsion forming surfactants. Exemplary embodiments employing a foaming agent may be used to provide a foam dispensing product which may be used, for example, to form a UV curable foam such as acrylated or polyurethane PGS. In some embodiments, foam dispensing provides for the creation of multi-part chemistries that react and set in situ after spraying which may allow, for example, endovascular resurfacing.

In one exemplary embodiment, a composition includes concentrate form components comprising Alcohol USP 56%, PGS 5%, Polawax A31 NF 1%, Cetomacrogol 1000-SO-(MH) 0.3%, Crodacol CS50-PA-(MH) 0.3%, Purified water 37.4% and in filled form components comprising: 55 g concentrate, 2.15 g Propellant A70. More generally, in accordance with some embodiments, a composition for delivery according to the disclosure comprises components in the following amounts, by weight, based upon the weight of the composition: from about 1% to about 50% PGS, and in some embodiments, from about 5% to about 25% PGS; from about 1% to about 100% solvent, and in some embodiments, from about 50% to about 75% Solvent; from about 0.1% to about 5% emulsifying agent, and in some embodiments from about 0.2% to about 3% emulsifying agent; from about 0.1% to about 5% surfactant, and in some embodiments, from about 0.2% to about 3% surfactant; from about 0.1% to about 5% active agent, and in some embodiments, from about 0.2% to about 3% active agent.

Delivery Articles and Compositions

Propellants

In accordance with various embodiments, spray and foam delivery articles may include one or more propellants. As used herein, propellants include but are not limited to compressed air and gas propellants. Of course, it will be appreciated that in other embodiments, propellants may be other than compressed gases. And in yet other embodiments, delivery articles may not involve pressure or propellants, and may include ingredients that form gel or ointment suspensions for the resorbable/degradable oligomer and polymer resins and optional actives, fillers and fibers.

The term “aerosol propellant” refers to and includes, for example, conventional propellants such as liquefied gases, usually naturally occurring hydrocarbons such as propane or butane, pentane, iso-pentane, and the like and compressed gases, such as, carbon dioxide, nitrogen, and air. In accordance with an exemplary embodiment, a propellant useful for compositions hereof comprises dimethyl ether. Generally, in accordance with the various embodiments, the compositions of the present invention may contain at least one propellant selected from the group consisting of C3 to C5 alkanes such as n-butane, isobutane, isopropane, and propane, dimethyl ether, C2-C5 halogenated hydrocarbons, 1,1-difluoroethane or hydroflurocarbon, difluoroethane, chlorodifluoroethane, chlorodifluoromethane, air (such as compressed air), nitrogen, carbon dioxide, and mixtures thereof.

In accordance with the various embodiments wherein the composition is provided in a propellant based sprayer comprising a chemical propellant, the composition comprises from about 3 to about 90% by weight, based on the total weight of the composition, and in some embodiments from about 3 to about 60%, by weight, or such as from about 3 to about 20% by weight, or such as from about 3 to about 10% by weight, or such as from about 3 to about 6%, by weight based on the total weight of the composition, including all ranges and subranges there between.

Other Additives: Surfactants/Stabilizers/Dispersants/Film Formers/Waxes

In accordance with some embodiments, other additives useful for compositions hereof include but are not limited to Surfactants, wetting agents, emulsifying agents, thickeners and rheology modifiers, film formers, moisturizers, and/or emollients, additives such as vitamins, sunscreen/UV protection, analgesics, anti-inflammatory, hemostatics, perfumes, hydrophobic barrier constituents, anti-microbial, biologic trophic agents, enzymes, fiber and combinations thereof, all by way of example.

In accordance with the various embodiments wherein the composition comprises one or more additives present from about 0.1% to about 5% by weight of each of one or more additives, wherein the total amount of additives present is from about 0.5% to about 10% by weight, based on the total weight of the composition.

Resorbable/Degradable Oligomers and Polymers: PGS and OGS Resins

In some exemplary embodiments according to the disclosure the compositions and articles comprise PGS resin. Generally, according to embodiments comprising PGS, the resin is prepared by known methods. In some examples, the PGS may be prepared according to U.S. Pat. No. 9,359,472, which provides water-mediated preparations of polymeric materials, including PGS and OGS. PGS is a simple glycerol-ester polymer created from the basic mammalian metabolites of glycerol and sebacic acid, both of which have a regulatory background with the FDA. Originally designed as a biodegradable polymer with improved elastic mechanical properties and biocompatibility, research on PGS-based medical applications has uncovered a number of unique properties that have bolstered its utility as a biomaterial. In addition to its elasticity, PGS demonstrates minimal swelling, undergoes surface degradation and exhibits mild acute and chronic inflammatory responses in vivo. Although the majority of researchers use the thermoset elastomer form of PGS, the polymer is customizable through a continuum of resin forms. PGS resin can be manufactured, according to the instant disclosure, as a soft gel, a lubricious paste, a sprayable or foamable composition, all of which avoid direct handling of what is otherwise a sticky tacky material that is difficult to handle. As further described herein, PGS is synthesized via a polycondensation reaction between glycerol and sebacic acid to first form a pre-polymer resin which is then converted into the thermoset elastomer.

PGS degrades primarily through hydrolysis of the ester linkage into smaller oligomers and ultimately to the starting monomers, glycerol and sebacic acid. PGS degradation is unique and differs from other resorbable polymers (e.g., polylactide, polyglycolide, and copolymers) in that PGS degrades via surface erosion as opposed to bulk erosion. The hydrolytic degradation of PGS into its component monomers, glycerol and sebacic acid, provides a resorbable material with high biocompatibility. Glycerol is a metabolic building block for lipids and has a long history of use in pharmaceuticals. Sebacic acid is the natural metabolite intermediate in ω-oxidation of medium and long-chain fatty acids. Further, co-polymers containing sebacic acid are used in chemotherapeutic drug delivery.

While embodiments of the invention are described primarily with respect to glycerol-sebacic acid ester compounds in both polymeric and oligomeric form, it is contemplated that the principles of the invention extend to other condensation oligomers and polymers of polyols and acids, including monoacids, diacids, free-fatty acids, and combinations thereof.

In some embodiments, the polyol component may be glycol, glycerol, erythritol, threitol, arabitol, xylitol, mannitol, sorbitol, maltitol, or combinations thereof. In some embodiments the acid may be sebacic acid, malonic acid, succinic acid, glutaric acid (5 carbons), adipic acid (6 carbons) pimelic acid (7 carbons), suberic acid (8 carbons), azelaic acid (9 carbons), cis-2-decenoic acid, cis-2-dodecenoic acid, cis-11-methyl-dodecenoic acid, 12 methyl-tetradecanoic acid, cis-9-ocatdecanoic acid, tetradecanoic acid, linoleic acid, oleic acid, palmitic acid, stearic acid, lauric acid, myristic acid, sapienic acid, cis-8-octadecenoic acid, cis-11-methyl-2-dodecenoic acid, or combinations thereof, with a preference for acids that demonstrate anti-microbial characteristics in the resultant polymer.

In some embodiments, long chain diacids are used having more than 10, more than 15, more than 20, or more than 25 carbon atoms. Non-aliphatic acids may be used. For example, versions of diacids having one or more double bonds may be employed to produce glycerol-diacid co-polymers. Amines and aromatic groups may also be incorporated into the carbon chain. Exemplary aromatic diacids include terephthalic acid and carboxyphenoxypropane. The diacids may also include substituents as well. Reactive groups such as amines and hydroxyls may increase the number of sites available for cross-linking. Amino acids and other biomolecules may modify the biological properties of the polymer. Aromatic groups, aliphatic groups, and halogen atoms may modify the inter-chain interactions within the polymer. Any condensation polymer formed from of any of the above listed or other polyols and any of the above listed or other acids may be included in compositions of the invention.

Examples of poly(polyol sebacate)s for use in the present invention include, but are not limited to, one or more of the following: poly(glycol-sebacate), poly(glycerol-sebacate), poly(erythritol-sebacate), poly(threitol-sebacate), poly(arabitol-sebacate), poly(xylitol-sebacate), poly(mannitol-sebacate), poly(sorbitol-sebacate), poly(maltitol-sebacate), and combinations thereof. In certain embodiments the poly(polyol-sebacate) is poly(glycerol-sebacate).

Examples of oligo(polyol-sebacate)s for use in the present invention include, but are not limited to, one or more of the following: oligo(glycol-sebacate), oligo(glycerol-sebacate), oligo(erythritol-sebacate), oligo(threitol-sebacate), oligo(arabitol-sebacate), oligo(xylitol-sebacate), oligo(mannitol-sebacate), oligo(sorbitol-sebacate), oligo(maltitol-sebacate), and combinations thereof. In certain embodiments the oligo(polyol-sebacate) is oligo(glycerol-sebacate).

Furthermore, while glycerol-sebacic acid ester compounds discussed herein are often referred to collectively as PGS, the glycerol-sebacic acid ester compound may be present in polymeric form, having a molecular weight greater than 10,000 as well as those having a molecular weight of 10,000 or less, which may be considered an oligomeric form (also referred to herein as OGS). Exemplary embodiments may employ some combination of high and low molecular weight forms of the polyester.

Condensation oligomers and polymers of a polyol and a diacid may also be characterized by the acid number (a measure of the number of carboxylic acid groups) and hydroxyl number (a measure of the number of hydroxyl groups). Acid number refers to a mass of potassium hydroxide (KOH) in milligrams that is required to neutralize one gram of condensation polymer or oligomer. Hydroxyl number refers to the number of milligrams of potassium hydroxide required to neutralize the acetic acid taken up on acetylation of one gram of condensation polymer or oligomer. Condensation oligomers useful in the invention typically have an acid number between about 50 mg/g and about 100 mg/g, about 55 mg/g and about 85 mg/g, about 55 mg/g and about 75 mg/g, or about 55 mg/g and about 65 mg/g; condensation polymers useful in the invention typically have an acid number between about 5 mg/g and about 55 mg/g, about 15 mg/g and about 50 mg/g, about 20 mg/g and about 50 mg/g, or about 35 mg/g and about 50 mg/g.

With respect to the mole ratio of polyol monomer to diacid monomer in a condensation polymer used in the present invention, such a mole ratio is typically about 1:1, though other ratios are within the scope of the invention. In some embodiments, the mole ratio of polyol monomer to diacid monomer can be about 1:0.8, about 1:1, about 1:1.2, about 1:1.5, about 1:2, about 1:3, about 1:4, or about 2:3.

Generally, PGS resin has an average molecular weight in the range of 1,000-50,000 Da; in some embodiments, the PGS resin has a molecular weight in the range of 15,000-25,000 Da. References herein to molecular weight refer to weight average molecular weight. For various applications contemplated hereunder, particularly for wound and surgical site applications, lower molecular weight PGS is preferred to take advantage of lower overall viscosities and shorter degradation times, particularly for in vivo applications, as compared with higher molecular weight forms.

Fillers; Micronized Thermoset PGS Filler

In some embodiments, as further described herein, the invention includes compositions comprising PGS that include a micronized thermoset PGS filler (also referred to as “PGS flour”) for forming constructs according to the disclosure. According to such embodiments, the sprayable formulations include a mixture of PGS comprising a resin of glycerol-sebacic acid ester, and thermoset PGS that has been processed into a flour or powder of fine particle size. Mixtures of PGS resin and micronized thermoset PGS is discussed in U.S. application Ser. No. 15/442,055 filed Feb. 24, 2017, which is hereby incorporated by reference. The filler of the composites in accordance with exemplary embodiments comprises thermoset PGS (or other polymer of a diacid and polyol) that has been processed into a flour or powder of fine particle size (e.g., less than 1000 microns). The PGS thermoset filler cross-link density is about 0.07 mol/L or greater, which is calculated with respect to the thermoset material prior to particularization by soaking samples in tetrahydrofuran for 24 hours to obtain a swollen mass, dried until a constant dry mass is acquired (typically about 3 days) and the swelling percentage is then used to calculate the crosslink density using the Flory-Rehner expression for tetra-functional affine networks.

Filler material comprising PGS flour has been shown by the inventors in the application referenced above to have unexpected and surprising benefits. Because PGS is a soft elastomer and thus would not ordinarily be considered a suitable filler material in many applications, particularly for dispersion within a matrix comprising PGS resin to form a composite that demonstrates significant differences in rheology and improved handling and processing characteristics over either PGS resin or thermoset PGS alone.

Filler particle size may vary depending on application, but the filler is generally between 0.5 and 1000 microns and typically less than 850 microns. Smaller particle sizes are generally preferred for additive manufacturing and traditional Brabender or fiber extrusion machines, with comparatively larger sizes being able to be used for industrial, orthopedic, wound care and dental applications. In some embodiments, maximum particle size is about 60 to 125 microns for additive manufacturing, while maximum particle size for other forms of extrusion is typically in the range of about 75 to about 300 microns, such as about 175 to about 250 microns.

The thermoset filler can be manufactured by any suitable method of forming fine particles of thermoset material. In one embodiment, thermoset PGS is processed into filler particles by cryogrinding. In this process, a sheet or other larger mass of thermoset PGS is frozen to very low temperatures, e.g. direct exposure to liquid nitrogen. This renders the PGS thermoset brittle enough to be ground into small granules while in its frozen state. The thus-formed filler particles resume their elastomeric state upon returning to ambient temperature after completion of the process. Cryogrinding may be most useful when filler particles having smaller diameters (e.g. about 300 microns or less) are desired.

In another embodiment, the filler particles are formed through an extraction and milling technique. PGS can be analogized to a sol-gel, with higher molecular weight chains acting as the gel and lower molecular weight chains acting as the connective sol. When thermoset PGS is soaked in an organic solvent, some sol portions are removed, which results in an unstable structure of gel portions capable of being ground into a fine powder.

In the extraction process of PGS filler manufacture, thermoset PGS is soaked in an organic solvent (such as ethyl acetate or THF) which dissolves a portion of the low molecular weight fractions of the PGS. This weakens the overall thermoset structure and allows it to crumble when agitated, such as with a dual-asymmetric centrifuge mixer, resulting in a fluffy powder-like material.

In some embodiments, ethyl acetate is a preferred organic solvent, as it has demonstrated better selectivity in dissolving low molecular weight fractions. Other organic solvents, such as THF, may also be used but can tend to also pull out some higher molecular weight fractions. The removal of some higher molecular weight fractions may be desired in some cases to produce smaller particle sizes. Particle size can be controlled based on solvent soak time, with longer soaks and/or removal of higher molecular weight fractions resulting in smaller particle sizes, as well as the glycerol to sebacic acid molar ratio used in the polymerization of PGS.

Regardless of the technique used, the resulting filler particles can then be further sized, for example, by sieving or other sizing techniques. The PGS filler particles are observed to be cohesive and tend to agglomerate. Accordingly, in some embodiments the filler particles can be wetted with ethyl acetate to reduce particle interaction as well as provide additional weight. Hydroxyapatite can also be used to prevent particle interactions by coating the particles, minimizing any interactions and resulting in a fine powder. In another embodiment, sizing may occur while the particles are in a harder, frozen state, such as under the presence of liquid nitrogen.

The molar ratio of glycerol:sebacic acid in the thermoset PGS used for the filler material may vary, but typically is in the range of 0.7:1 to 1.3:1. Reducing the amount of glycerol relative to the amount of sebacic acid produces a larger amount of finer particle sizes during filler particle production using the extraction method due to a smaller percentage of sol holding the structure together. However, higher amounts of glycerol, for example, up to 1.3:1 glycerol:sebacic acid, is also suitable, with a preference in some embodiments for a 1:1 molar ratio. While the stoichiometric ratios of glycerol to sebacic acid can be varied for the PGS particles, the particles should still be of a surface energy similar to that of the resin. In some embodiments, that is accomplished by providing the PGS filler particles having a molar ratio of glycerol:sebacic acid that is similar or the same as that of the resin in the composition. In a presently preferred embodiment, a composite includes a PGS thermoset filler made from 1:1 glycerol:sebacic acid molar ratio dispersed in a PGS resin that also has a 1:1 glycerol:sebacic acid molar ratio.

As with the PGS resin material, the polymeric material used to form the filler particles may be doped with an active ingredient.

Generally, the composite is about 10% by weight to about 90% by weight filler. In some embodiments, the composite is no more than 50% by weight or more filler (for 75-250 μm particles), and is in the various embodiments present in a lower percentage than the resin to achieve a compositional viscosity suitable for delivery via spray or foaming dispensing methods.

In some embodiments, sprayed constructs formed using the PGS flour with the resin may be cured prior to or after application to a substrate, such as tissue, and more particularly wound or surgical site tissue. For example, in some embodiments, the construct is formed by dispensing on an inert substrate prior to application to the target, such as tissue, and is cured after dispensing and prior to tissue application. In other embodiments, the sprayed constructs may be formed in situ and need not be cured. In yet other embodiments, the sprayed constructs may be formed in situ and cured by a method that is suitable for the construct, such as, for example, UV light cured, heat cured, chemically cured, or other methods.

The particulate PGS, herein referred to as PGS flour, may vary in size depending on application, but the flour particles are generally between 0.5 and 1000 microns and typically less than 850 microns. In some embodiments, average particle size ranges from about 75 to about 300 microns, such as about 175 to about 250 microns. In some embodiments, a composition is formed in an initial mixture of about 20% to about 70% by weight glycerol-sebacic acid ester and about 30% to about 80% by weight PGS flour to create a composition capable of processing by extrusion processes such as with a twin-screw extruder to form defined shapes such as sheet, rod, tube or other shape defined by the die construction. Embodiments which include the thermoset PGS powder particles results in in situ pore formation as a result of two-phase degradation due to the resin and flour mismatched crosslink density.

The molar ratio of glycerol: sebacic acid in thermoset PGS flour may vary, but in some embodiments is in the range of 0.7:1 to 1:1. The particles can be manufactured, for example, by grinding thermoset PGS or an extraction and milling technique by soaking in an organic solvent (such as ethyl acetate or THF) to dissolve a portion of the low molecular weight fractions in the PGS. The solvent weakens the overall thermoset structure and allows it to crumble when agitated, such as with a dual-asymmetric centrifuge mixer, resulting in a fluffy powder-like material. As with the PGS resin, the PGS flour particles themselves may also contain active ingredients.

In some embodiments, fillers are also selected from the group consisting of inorganic salts, calcium phosphate, hydroxyapatite, β-Tricalcium phosphate, titanium dioxide, collagen, gelatin, PCL, PGLA, PGA, PLA and combinations thereof.

In accordance with the various embodiments wherein the composition comprises one or more of PGS flour and other powders or fillers, each filler is present from about 1% to about 50% by weight, wherein the total amount of filler present is from about 10% to about 50% by weight, based on the total weight of the composition.

Solvents

In accordance with the various embodiments, solvents useful for compositions hereof include but are not limited to ethanol methanol, 1-propanol, 2-propanol, pentane, 1-butanol, 2-butanol, ethyl formate, isopropylacetate, isobutyl alcohol, isopropyl acetate, butyl acetate, propyl acetate, DMSO, ethyl acetate, acetone

In accordance with the various embodiments wherein the composition comprises a solvent, present from about 20% to about 80% by weight, based on the total weight of the composition.

Biological Actives and other Additives

The specific trophic agents, growth factors and other materials used in the initial composition may vary and can be formulated for depending on the wound condition and underlying pathology sought to be addressed. Accordingly, in some embodiments the composition can be formulated for local wound care factors selected from the group consisting of absorption of polymer, promotion of cell migration and infiltration, space filling, wound and skin sealing, burned tissue coverage, cell seeding, and combinations thereof.

The active ingredient and delivery article for spray embodiments may be a combination of PGS and one or more of an antibiotic, nutrient, colloid, hydrogel, film former, collagen, and drug, for example. In some embodiments compositions of the present invention further include a bioactive material. Such a bioactive material can be a vitamin, such as vitamin E or C, for example, mineral, and/or include tocopherol, ascorbate, retinoic acid, or combinations thereof. Alternatively, the bioactive material can be cells, such as stem cells, progenitor cells, mesenchymal cells, trophic cells, somatic cells, or combinations thereof. In other embodiments, the bioactive material may be biologically active short peptide sequences, growth factors, proteoglycans, glycoproteins, glycosaminoglycans and polysaccharides, nutrients, cytokines, hormones, angiogenic factors, immunomodulatory factors, drugs, or combinations thereof.

Surfactants (including wetting agents, moisturizers, and/or emollients), film formers, rheology modifiers, with additives such as vitamins, sunscreen/UV protection, analgesics, anti-inflammatory agents, hemostatics, perfumes, hydrophobic barrier constituents, antimicrobials, biologic trophic agents, enzymes, fiber and combinations thereof, all by way of example. For embodiments in which the composition is sprayed as a string foam, an agent to promote fiber-foaming or poration for closed or open cell expansion of string resin may also be incorporated that can aid in the formation of an interlocking network of fiber elements.

In accordance with the various embodiments wherein the composition comprises one or more of biological actives, each active is present from about 0.1% to about 50% by weight, wherein the total amount of active present is from about 0.5% to about 25% by weight based on the total weight of the composition.

Weight Amounts of Ingredients

The compositions according to the disclosure include ingredients that are present in amounts by weight, typically based on the weight of the composition, wherein the ingredients may be present at a concentration, by weight, of from about 0.1% to about 100%. Thus, in various embodiments, an ingredient may be present in a composition in a weight percent amount from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99 percent by weight, including increments and ranges thereof and there between.

### EXAMPLES

Wound care study with compositions according to the disclosure.

Test articles include: uncured PGS resin and uncured OGS resin, all applied to wound tissue in situ using a hand-held dispenser.

A standard biopsy punch wound model was performed to evaluate the described test articles within a wound bed.

Two full dermal thickness 8 mm wounds were created on Sprague Dawley rats. A onetime application of test articles at wound creation were applied and wrapped with a secondary dressing. Test articles included PGS resin and OGS resin.

An untreated sham group was also wrapped with no treatment as a control.

All treatment groups were unwrapped and imaged at day 3, 7, 10 and 14 and macro wound measurements were taken. At each unwrap time point a set of animals for each treatment group were euthanized for histological analysis and measurement of wound size.

Results:

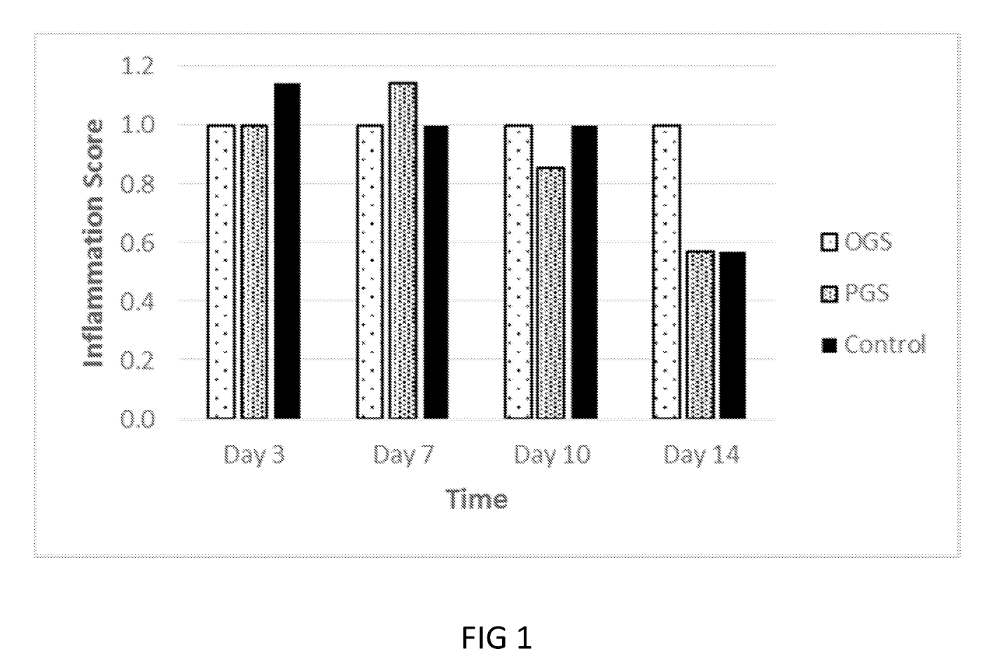

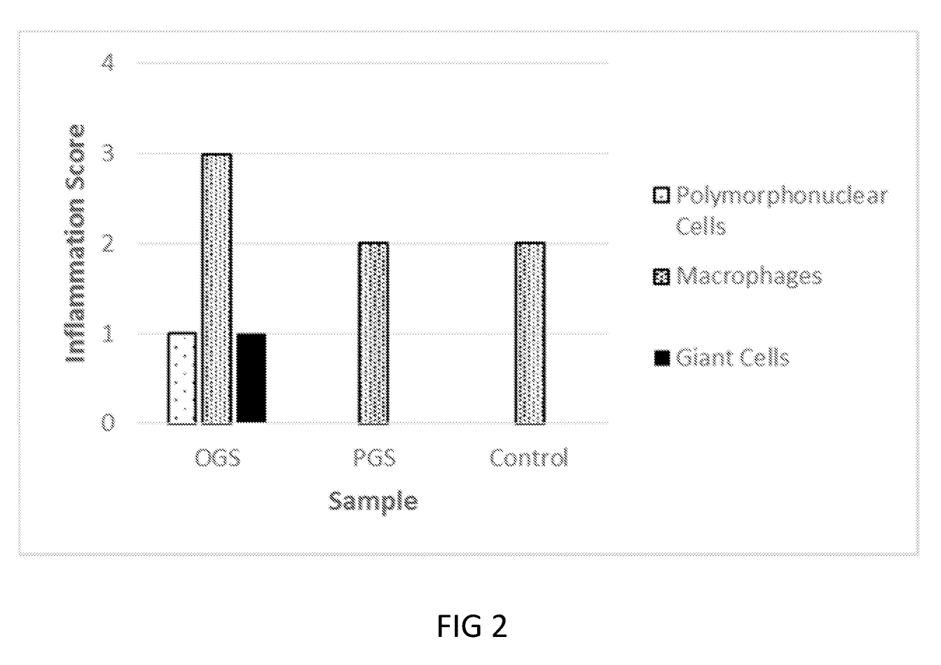

Inflammation was scored by a trained histopathologist per the following scale: 0-absent, 1-Minimal, 2-Mild, 3-Moderate, 4-Marked. Referring now to the drawings, FIG. 1 provides a graphical representation of inflammation scores of OGS resin, PGS resin and Control samples at Day 3, 7, 10 and 14 of the study. FIG. 1 shows that the inflammatory response of both resins track the Control sample out to Day 10 with the PGS resin showing a slight decrease in inflammation with respect to OGS at Day 14. Referring again to the drawings, FIG. 2 provides a graphical representation of individual inflammation markers for OGS resin, PGS resin and Control samples at Day 14 of the study. The trend is further supported by the individual markers of inflammation as seen in FIG. 2 as the PGS resin trends as the control.

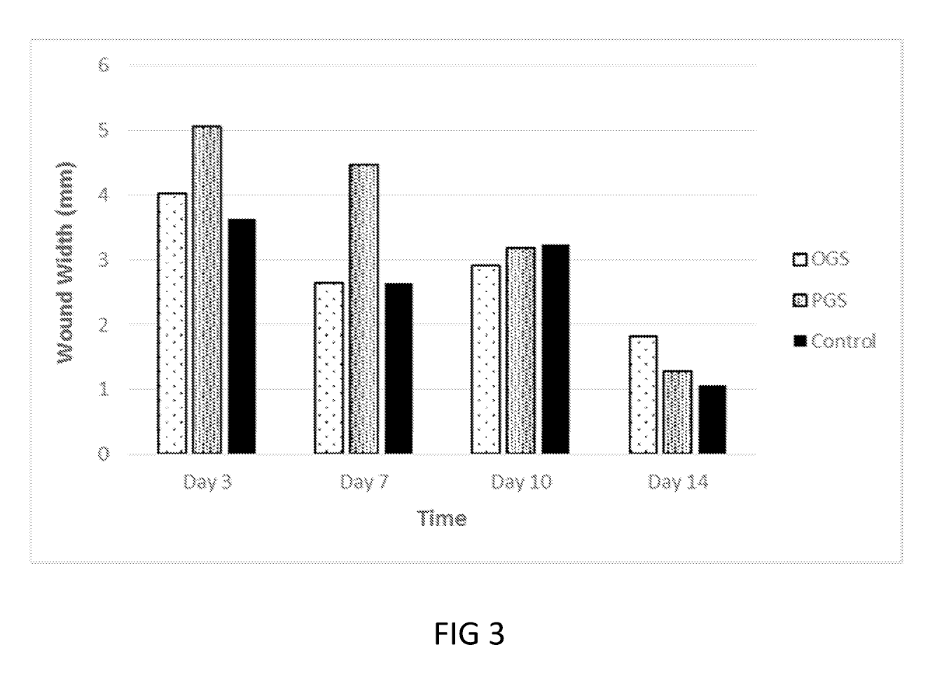

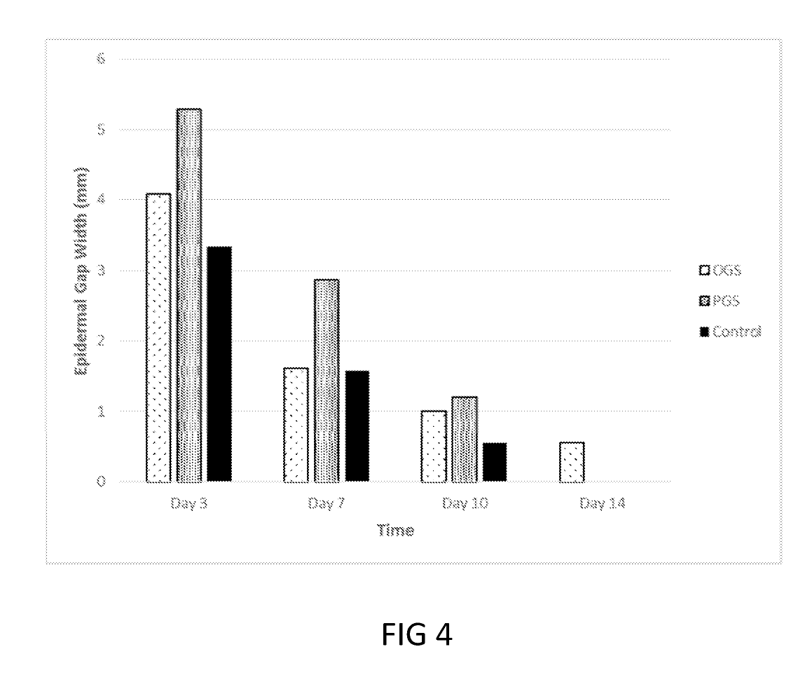

Further indication of OGS and PGS resins supporting wound healing are illustrated by measurement of healing wound. Referring again to the drawings, FIG. 3 provides a graphical representation of Wound Width measurements for OGS resin, PGS resin and Control samples at Day 3, 7, 10 and 14 of the study, and FIG. 4 provides a graphical representation of Epidermal Gap Width measurements for OGS resin, PGS resin and Control samples at Day 3, 7, 10 and 14 of the study. At Day 14 the width for both PGS resin and Control samples was 0. Each of FIG. 3 and FIG. 4 shows that wound width and epidermal gap distance, respectively, for the OGS resin and PGS resin samples follow the same rate of wound closure as the Control sample. Again, the PGS resin shows a slight benefit over the OGS resin. Without being bound by any particular theory it is believed that the composition differences of the low molecular weight fractions between the OGS and PGS resins are causative of the improved healing observed in the PGS resin samples.

While exemplary embodiments described herein are primarily directed to wound care and other anatomical applications, the invention is not so limited. Exemplary embodiments can also be used, for example, to make controlled release pods or mats for industrial controlled release materials in the field, controlled release dispensing for environmental applications of biocides/pesticides and the like, and environmental containment when formulated with an absorbant.

It to be understood that, as used herein the terms “the,” “a,” or “an,” mean “at least one,” and should not be limited to “only one” unless explicitly indicated to the contrary. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity. Thus, for example, reference to “a portion” includes examples having two or more such portions unless the context clearly indicates otherwise.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

The compositions and methods according to the present disclosure can comprise, consist of, or consist essentially of the elements and limitations described herein, as well as any additional or optional ingredients, components, or limitations described herein or otherwise known in the art. While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

It will be apparent to those skilled in the art that various modifications and variations can be made in the composition and methods of the invention without departing from the spirit or scope of the invention. Thus, it is intended that the present invention cover the modifications and variations of this invention provided that they come within the scope of the appended claims and their equivalents. It should be understood that all patents and published patent applications referenced are incorporated herein in their entireties.

What is claimed is:

1. An article comprising
(1) a dispenser selected from a pressurized and a pressurizable apparatus, and
(2) a composition comprising
(a) at least one resin selected from PGSU resin, PGS resin selected from PGS and OGS, and other polyol/diacid resins, and
(b) at least one solvent,
wherein the composition (2) is contained within the dispensing unit.

2. An article according to claim 1, wherein the resin has a molecular weight in the range of from about 1,000 to about 50,000 Da.

3. An article according to claim 1, comprising (c) at least one propellant or other dispersant.

4. An article according to claim 1, comprising (d) at least one biological active selected from biologic tissue engineering ECM-compatible biologic components, antimicrobials, drugs, growth enhancers, stimulants, trophic agents, tissues, tissue matrices, and cells.

5. An article according to claim 1, comprising (e) at least one of structural matrix materials, fibers, fillers, gelatin and collagen.

6. An article according to claim 5, wherein (e) comprises a filler comprising micronized PGS thermoset resin.

7. An article according to claim 1, comprising one or more additional additives (f).

8. An article comprising
(1) a dispenser, and
(2) a composition comprising
(a) at least one PGS resin selected from PGS and OGS, and
(b) at least one solvent,
(c) at least one propellant or other dispersant, and
(e) one or more additional additives,
wherein the composition (2) is contained within the dispensing unit.

9. An article according to claim 9, wherein (a) comprises PGS resin present from about 25 to about 40% by weight, (b) is present from about 60% to about 75% by weight solvent, (c) is present from about 10% to about 60%, and (e) comprises at least one surfactant, present at about 4% or less by weight, and from about 3 to 10% by weight other additives selected from surfactants, wetting agents, moisturizers, emollients, film formers, rheology modifiers; vitamins, sunscreens, analgesics, anti-inflammatory agents, hemostatics, perfumes, hydrophobic barrier constituents, antimicrobials, biologic trophic agents, enzymes, fibers and combinations thereof.

10. An article comprising
(1) a dispenser, and
(2) a composition comprising
(a) at least one solvent, and
(b) a filler comprising micronized PGS thermoset resin,
wherein the composition (2) is contained within the dispensing unit.

11. An article according to claim 10, comprising (c) at least one propellant or other dispersant,

12. An article according to claim 10, comprising (d) at least one biological active selected from biologic tissue engineering ECM-compatible biologic components, antimicrobials, drugs, growth enhancers, stimulants, trophic agents, tissues, tissue matrices, and cells.

13. The article of claim 10 further comprising another filler (e) selected from the group consisting of inorganic salts, calcium phosphate, hydroxyapatite, β-Tricalcium phosphate, titanium dioxide, collagen, gelatin, PCL, PGLA, PGA, PLA and combinations thereof.

14. An article comprising a conformable string or fiber-like extrudate composite comprising an PGS or OGS resin having a molecular weight of from about 5,000 to about 50,000 Da and a molar ratio of glycerol to sebacic acid in the range of 0.7:1 to 1.3:1, and at least one additive selected from surfactants, wetting agents, moisturizers, emollients, film formers, rheology modifiers; vitamins, sunscreens, analgesics, anti-inflammatory agents, hemostatics, perfumes, hydrophobic barrier constituents, antimicrobials, biologic trophic agents, enzymes, fibers and combinations thereof.

15. An article according to claim 14, further comprising at least one biological active selected from biologic tissue engineering ECM-compatible biologic components, antimicrobials, drugs, growth enhancers, stimulants, trophic agents, tissues, tissue matrices, and cells.

16. An article according to claim 14, wherein the resin is either cured by one of UV or chemical curing or uncured.

17. An article comprising a conformable string or fiber-like extrudate composite comprising an PGS or OGS resin having a molecular weight of from about 1,000 to about 50,000 Da, at least one additive selected from a surfactants, wetting agents, moisturizers, emollients, film formers, rheology modifiers; vitamins, sunscreens, analgesics, anti-inflammatory agents, hemostatics, perfumes, hydrophobic barrier constituents, antimicrobials, biologic trophic agents, enzymes, fibers and combinations thereof.
and a micronized PGS thermoset filler having a particle size between 0.5 and 1000 microns, the thermoset filler is present from about 40% by weight to about 70% by weight of the composite and has a cross-link density of about 0.07 mol/L or greater, and the thermoset filler and the resin each have a molar ratio of glycerol to sebacic acid in the range of 0.7:1 to 1.3:1.

18. An article according to claim 17, further comprising at least one biological active selected from biologic tissue engineering ECM-compatible biologic components, antimicrobials, drugs, growth enhancers, stimulants, trophic agents, tissues, tissue matrices, and cells.

19. An article according to claim 17, wherein the resin is either cured by one of UV and chemical curing or uncured.

20. A method of forming an article a conformable string or fiber-like extrudate composite, the method including the steps comprising
(1) providing a pressurized dispenser containing a composition comprising
(a) at least one PGS resin selected from PGS and OGS, and
(b) at least one solvent,
(c) at least one propellant or other dispersant, and
(e) one or more additional additives,
(2) directing a spray nozzle affixed to the dispenser toward a target substrate,
(3) actuating the dispenser to extrude the composition on the target substrate,
(4) tracking the spray nozzle along a path that directs the extruded composition in a predetermined pattern over the target substrate to a selected thickness,
(5) suspending the actuation of the spray nozzle,
(6) repeating one or more of the above steps (2)-(5).
